(19) United States
(12) Patent Application Publication
(10) Pub. No.: US 2025/0303091 A1
(43) Pub. Date: Oct. 2, 2025

(54) APPARATUS AND METHOD FOR REDUCING ETT SECUREMENT DEVICE RELATED PRESSURE INJURY

(71) Applicant: Securisyn Medical, LLC, Littleton, CO (US)

(72) Inventor: Arthur Kanowitz, Fountain Hills, AZ (US)

(73) Assignee: Securisyn Medical, LLC, Littleton, CO (US)

(21) Appl. No.: 19/035,696

(22) Filed: Jan. 23, 2025

Related U.S. Application Data
(60) Provisional application No. 63/624,242, filed on Jan. 23, 2024.

(51) Int. Cl. A61M 16/04

(52) U.S. Cl. CPC A61M 16/0497; A61M 2209/088; A61M 2210/0606

(57) ABSTRACT

An airway device securement and stabilization system for maintaining an airway device in a preselected position in a patient's trachea while providing a mechanism for easy, rapid, and simultaneous movement of both the airway device and a pressure reduction cushion to different positions on the patient's face relative to the patient's oral cavity, thereby relieving pressure from the airway device and the securement and stabilization system on the patient's upper lip to minimize the risk of pressure injuries thereto.

### PRIORITY TO RELATED PATENT APPLICATIONS

This application is a U.S. patent application which claims the benefit of U.S. Provisional Patent Application Ser. No. 63/624,242 filed on Jan. 23, 2024, which is incorporated herein by reference.

### FIELD OF THE INVENTION

The present invention relates generally to medical devices. Specifically, the present invention relates to an airway securement and stabilization system designed to maintain an airway device in a preselected position in the trachea of a patient while also preventing any pressure injuries to a patient which could be caused by the system's securement device. More specifically, the present invention relates to an airway securement and stabilization system having a supporting cushion or foam apparatus for preventing pressure injuries to a patient's upper lip that also provides an easy and rapid position adjustment mechanism for moving the supporting cushion or foam to different positions on a patient's upper lip, thereby relieving pressure thereon and minimizing the risk of pressure injuries.

### BACKGROUND OF THE INVENTION

Endotracheal intubation is a medical procedure used to place an airway device (artificial airway) into a patient's trachea or airway. The use of an airway device is mandated in situations where an individual, or an animal in veterinary applications, is unable to independently sustain the natural breathing function or maintain an open airway due to unconsciousness, trauma, disease, drugs or anesthesia. Thus, life-saving mechanical ventilation is provided through the airway device, which may be in the form of an endotracheal tube (ETT), or a supraglottic airway device such as a laryngeal mask airway (LMA), King Airway, or one of several other commercially available airway devices.

Endotracheal intubation is accomplished by inserting an airway device into a patient's mouth, down through the throat and larynx, and into the trachea. This procedure creates an artificial passageway through which air can freely and continuously flow in and out of a patient's lungs and prevents the patient's airway from collapsing or occluding.

It is very important that the airway device be positioned correctly and maintained in the correct position in the trachea. If the device moves out of its proper position in the trachea and into the right main stem bronchial tube, the left lung will not be ventilated properly, leading to atelectasis and associated pulmonary complications, while the right lung will be over ventilated leading to tension pneumothorax. Moreover, if the airway device moves completely out of the trachea and into the pharynx, esophagus or completely outside the body (unplanned extubation), the patient will become hypoxic due to the lack of ventilation to the lungs, a condition which typically results in life-threatening brain injury or death within a matter of only a few minutes.

Even after an airway device has been positioned correctly, subsequent movement of the patient can lead to inadvertent movement of the device and unplanned extubation of the patient, as hereinabove described. An intubated patient may restlessly move about and may also attempt to forcibly remove an airway device, whether conscious or subconscious, particularly if the patient is uncomfortable or having difficulty breathing, which can lead to panic.

Unintentional movement of the airway device is not uncommon, particularly when the patient is moved from an out-of-hospital setting such as from the scene of an accident to an emergency department of a hospital. Further, anytime an intubated patient is be moved, for example, not only from an ambulance to a trauma facility, but also from one hospital to another hospital, from one area of the hospital to another area in the same hospital (imaging, laboratory, operating theater), or from a hospital to an outpatient rehabilitation facility, unintentional movement of an airway device is a risk.

Recent advances in the field have led to the successful development of airway securement and stabilization systems which may be easily, efficiently, and quickly fitted to and removed from any airway device within a range of sizes that may be used with human patients to maintain an airway in a patient's trachea. The securement and stabilization system prevents clinically significant movement of the airway device with respect to a patient's vocal cords in response to the application of forces in any direction to the device. However, obtaining adequate securement requires application of a securement device in such a manner that forces are applied to the tissues of the face, lips and oral cavity. The applied force can lead to decreased capillary blood flow and decreased tissue perfusion, which can create a cascade of increasing tissue ischemia, tissue injury, and eventual tissue infarction (ulcer). The amount (magnitude) of applied force combined with the length of time the force is applied, will determine the severity of the tissue injury.

A Medical Device Related Pressure Injury (MDRPI), a subcategory of Hospital Acquired Pressure Injuries (HAPI), is defined as “a localized injury to the skin and/or mucous membranes, with a history of an external medical device at the location of the injury and mirrors the shape of the device.” Airway device related pressure injuries are MDRPI's specifically related to either the ETT applying pressure on the tissue of the oral cavity and lips or the ETT stabilizer causing pressure on the lips or face. The tissue hypoxia caused by the decreased capillary blood flow can manifest as tissue redness. As the cascade progresses to tissue infarction, the injury is manifest by tissue ulceration. Pressure injuries can be painful and debilitating to the patient and advanced stage pressure injuries can include a prolonged course to healing and cause permanent scarring. In addition to the significant morbidity associated with pressure injuries, they can also be very costly to healthcare systems. In October 2008, the Center for Medicare and Medicaid Services (CMS) instituted a policy to withhold reimbursement to acute care hospitals for the costs of treating hospital-acquired conditions including pressure injuries. Stage 3 (full-thickness skin loss) and Stage 4 (full-thickness skin loss and tissue loss) pressure injuries are considered “never events” and are included in the category list of Hospital Acquired Conditions nonreimbursable by CMS. The change to place responsibility of hospital acquired pressure injuries on the admitting hospital provides increased incentive for hospitals to identify at-risk patients and implement preventive measures. In addition, The Joint Commission considers the development of Stage 3 and Stage 4 pressure injuries during hospitalization as patient safety sentinel events. A sentinel event is an unexpected event in a healthcare setting that results in serious injury or death. Sentinel events are named as such because they signal the need for immediate investigation and response.

In a 2022 systematic review and meta-analysis of airway device related injuries, 12 studies representing 9,611 adult patients and 152 pediatric patients, the incidence of endotracheal tube related pressure injury was 4.2%. In the same study, the endotracheal tube anchoring device was found to reduce the risk of pressure injury from the tube itself by about half (risk ratio 0.54; range 0.38-0.77). ETT fixator device-related pressure injuries have been shown to be present in as many as 58% of patients who have had their ETT's secured with tape and as high as 9% in patients who have had their ETT's secured with a commercial fixator device. The risk of pressure ulcers from ETT securing devices increases by a factor of six when vasopressors are used.

Frequent side to side repositioning of the ETT can decrease the incidence of lip pressure injuries. Silicone-based thin foam dressings used as a barrier between the skin and ETT fastener can prevent the development of facial ulcers. Any hospital-acquired pressure injury can result in significant care costs, depending on the type and stage of the injury. Because medical devices such as endotracheal tubes are so ubiquitous in healthcare, costs associated with these specific pressure injuries in themselves are significant, even by historic standards. For example, a 2010 cost analysis of intubation-related tracheal injury by Bhatti, et al. found that among 3,232 discharge records which included treatment for tracheal injury from an endotracheal tube, length of stay was longer, averaging 4.7 days and cost an average $11,025 per patient discharge. A cost analysis by Padula found that hospital acquired pressure injuries could cost, on average, $10,708 per patient.

It has been observed that the weight of the ventilator circuit, which includes the airway device, a 15 mm connector, and an airway operatively connected to a source of ventilatory air, may apply rotational forces or torque to the securement and stabilization system. The torque results in a realignment or shifting of the normally evenly distributed pressure applied to the patient's upper lip by portions of the securement and stabilization system to be more concentrated on a smaller surface area of the patient's upper lip. The concentrated force increases the risk of pressure related injuries to the patient's skin and underlying tissues, complications which may lead to prolonged hospitalization, may increase the risk of morbidity and mortality, and may contribute to increased healthcare costs.

In view of the foregoing, it will be apparent to those skilled in the art from this disclosure that a need exists for an apparatus which provides adequate airway device tube securement and stabilization but also provides a mechanism and technology to minimize the magnitude of and the time during which the device and securement apparatus exert pressure on any specific region of the patient's upper lip. The present invention addresses these needs in the art as well as other needs, all of which will become apparent to those skilled in the art from the accompanying disclosure.

### SUMMARY OF THE INVENTION

To address the aforementioned needs in the art, an adjustable airway device securement and stabilization apparatus for securing and stabilizing an airway device to maintain an airway to a patient's lungs, the patient having a face, a mouth, an upper and a lower lip, a nose, an oral cavity, a throat, vocal cords or larynx, a trachea having a length and forming an air passageway or airway in the patient, and a carina defining a point at which the trachea bifurcates into a left and a right bronchial tube. The airway device securement and stabilization apparatus further includes an upper lip cushioning member and an integrated cushioning member repositioning apparatus is provided to minimize the risk of pressure injuries to any specific region of the patient's upper lip on which the airway device and securement apparatus exerts pressure. The upper lip cushioning member is mounted on a selectively movable attachment mechanism or support member operatively connected to the airway securement and stabilization apparatus and adapted to selectively and simultaneously reposition the cushioning member and the airway device on the patient's upper lip. The selectively movable support member includes a carriage or shuttle adapted to easily reposition the upper lip cushioning member to different positions across the patient's upper lip (i.e., left, center and right), thereby rotatably shifting the applied pressure across at least three positions and relieving the pressure applied to tissue under each position for selected periods of time, thereby minimizing the risk of pressure injuries thereto.

In an embodiment, the carriage or shuttle of the cushioning member repositioning apparatus is adapted to slide over a track operatively connected to the securement and stabilization apparatus.

In another embodiment, the carriage or shuttle includes a releasable attachment mechanism adapted to secure the cushioning member repositioning apparatus in a preselected position along the track.

In yet another embodiment, the upper lip cushioning member is a cushion or pillow formed of resilient cushioning material compatible with medical applications.

In an embodiment, the cushion or pillow may be formed of a wide selection of materials including foam, silicone, etc. or with air or liquid inside to allow for pressure to be redistributed with the pillow as it is moved to different positions.

In still another embodiment, the upper lip cushioning member is a split pillow member.

In another embodiment, the upper lip cushioning member is a rectangular block shaped pillow.

In yet another embodiment, the carriage or shuttle of the cushioning member repositioning apparatus is operatively connected to an airway device securement and stabilization apparatus, the apparatus including a supporting rail or frame adapted to be releasably secured to a patient, an interlock collar operatively connected to the airway device, a generally cylindrically-shaped tower structure or clamshell-type clamping member operatively connected to the rail, the clamping member interacting in a clamping engagement with a continuous sidewall of the airway device via the interlock collar.

These and other features, aspects and advantages of the present invention will become apparent to those skilled in the art from the following detailed description of preferred embodiments taken in connection with the accompanying drawings, which are briefly summarized below, and by reference to the appended claims.

### BRIEF DESCRIPTION OF THE DRAWINGS

Referring now to the attached drawings which form a part of this original disclosure:

FIG. 1 is a side perspective view of an airway device securement and stabilization system including pressure injury reduction technology in the form of an upper lip cushioning member and an integrated cushioning member repositioning apparatus in accordance with an embodiment of the present invention;

FIG. 2 is a side perspective view of an interlock collar portion of the airway device securement and stabilization system having an airway device positioned therein;

FIG. 3 is a top view of an airway device securement and stabilization system of FIG. 1 having an interlock collar and airway device releasably and securely positioned therein;

FIG. 4 is a top perspective view of an exemplary layer of cushioning foam in accordance with an embodiment of the present invention;

FIG. 5 is a top perspective view of a rail portion of the airway device securement and stabilization system in accordance with an embodiment of the present invention;

FIG. 6 is a rear elevation view of the airway device securement and stabilization system of FIGS. 1 and 3;

FIG. 7 is a side elevation view of the airway device securement and stabilization system of FIGS. 1 and 3;

FIG. 8 is a side perspective view of the repositioning apparatus of FIGS. 1-7 with the supporting rail removed to better show the elements of the repositioning apparatus;

FIG. 9a is a top view of the airway device securement and stabilization system of FIGS. 1-5 illustrating the airway device and an upper lip cushioning member in a centered position;

FIG. 9b is a top view of the airway device securement and stabilization system of FIGS. 1-5 illustrating the airway device and an upper lip cushioning member repositioned to the left;

FIG. 9c is a top view of the airway device securement and stabilization system of FIGS. 1-5 illustrating the airway device and an upper lip cushioning member repositioned to the right;

FIG. 10a is a top view of the airway device securement and stabilization system of FIG. 9b illustrating the footprint of an upper lip cushioning member repositioned to the left;

FIG. 10b is a top view of the airway device securement and stabilization system of FIG. 9a illustrating the footprint of an upper lip cushioning member in a centered position;

FIG. 10c is a top view of the airway device securement and stabilization system of FIG. 9c illustrating the footprint of an upper lip cushioning member repositioned to the right;

FIG. 10d is a top view of the airway device securement and stabilization system of FIGS. 9a-9c illustrating the all-position footprints of an upper lip cushioning member;

FIG. 11 is a side perspective view of an airway device securement and stabilization system having an airway device releasably and securely positioned therein showing a single or solid foam embodiment of the upper lip cushioning member;

FIG. 12 is a side perspective view of an airway device securement and stabilization system in accordance with another embodiment of the present invention;

FIG. 13 is a rear side perspective view of the airway device securement and stabilization system shown in FIG. 12 illustrating an upper lip cushioning member and attachment mechanism secured thereto;

FIG. 14 is a front perspective view of the airway device securement and stabilization system shown in FIGS. 12 and 13;

FIG. 15 is a front perspective view of a pillow or cushion assembly operatively connected to an attachment mechanism or shuttle in accordance with an embodiment of the present invention;

FIG. 16 is a rear perspective view of the pillow or cushion assembly operatively connected to an attachment mechanism or shuttle shown in FIG. 15;

FIG. 17 is a front perspective view of the pillow or cushion shown in FIGS. 15 and 16;

FIG. 18 is a rear perspective view of the pillow or cushion shown in FIG. 17;

FIG. 19 is a front perspective view of the attachment mechanism or shuttle of the pillow or cushion assembly of FIG. 15;

FIG. 20 is a rear perspective view of the attachment mechanism or shuttle shown in FIG. 19;

FIG. 21 is a front perspective view of a pillow or cushion in accordance with another embodiment of the present invention;

FIG. 22 is a rear perspective view of the pillow or cushion of FIG. 21;

FIG. 23 is a rear perspective view of a pillow or cushion formed of a single piece of foam, silicone, or other resilient material in accordance with an embodiment of the present invention;

FIG. 24 is a front perspective view of the pillow or cushion of FIG. 23;

FIG. 25 is a front perspective view of a pillow or cushion having a truncated pyramid-shaped body in accordance with an embodiment of the present invention; and

FIG. 26 is a rear perspective view of the pillow or cushion of FIG. 25.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

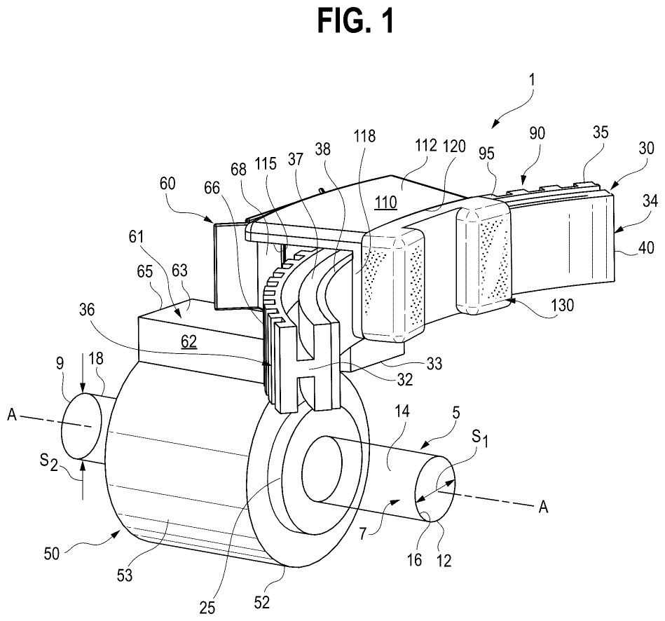

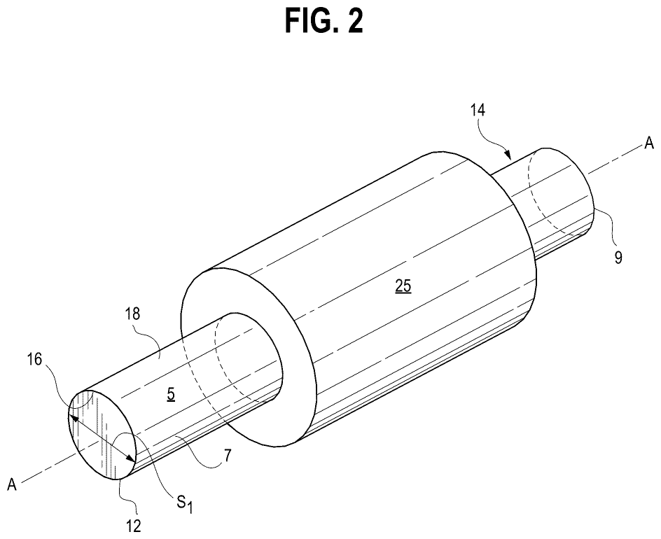

Referring initially to FIG. 1, portions of an adjustable airway device securement and stabilization system, referred to herein for purposes of brevity as “the stabilization system”, are depicted generally at 1 in accordance with an embodiment of the present invention. The stabilization system is used to maintain an airway in a human patient under conditions where natural respiration is impossible or severely compromised. The system is illustrated in operative securing engagement with an airway device, by way of example and not of limitation, an endotracheal tube depicted generally at 5. The airway device has a flexible elongate body 7 extending along a longitudinal axis A-A and having a length, an internal diameter S₁ and an external diameter S₂, a distal or machine end portion 9, a proximal or patient end portion 12, and a continuous sidewall 14 having an internal surface 16 and an external surface 18 extending between the proximal and the distal ends, thereby forming a hollow conduit or airway. As shown in FIG. 1 and in greater detail in FIG. 2, an adjustable interlock collar 25 is selectively positioned on and in operative securing engagement with the airway device. As described below, the airway device and the interlock collar are adjustably held in position by a securing apparatus or stabilizer in the form of a generally cylindrically-shaped tower structure or clamshell-type clamping member 50 which allows for depth adjustment of the airway device upon insertion in a patient to establish and maintain an air passageway to a patient's lungs via the patient's mouth, oral cavity, throat, past a patient's vocal cords or larynx into a patient's trachea and to a patient's carina (the point where the trachea bifurcates into the bronchial tubes) for respiration of the patient.

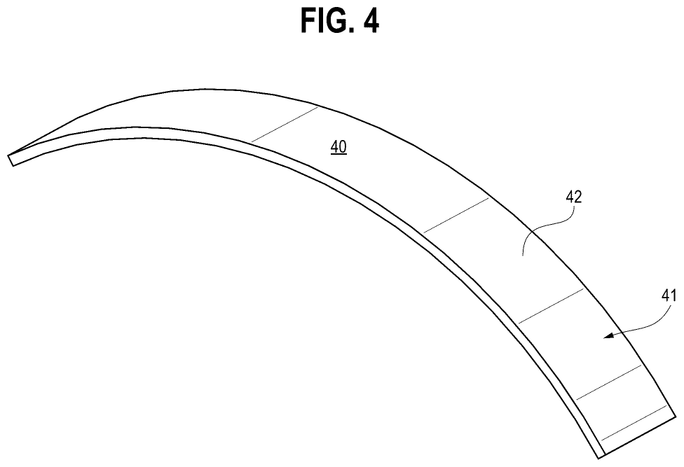

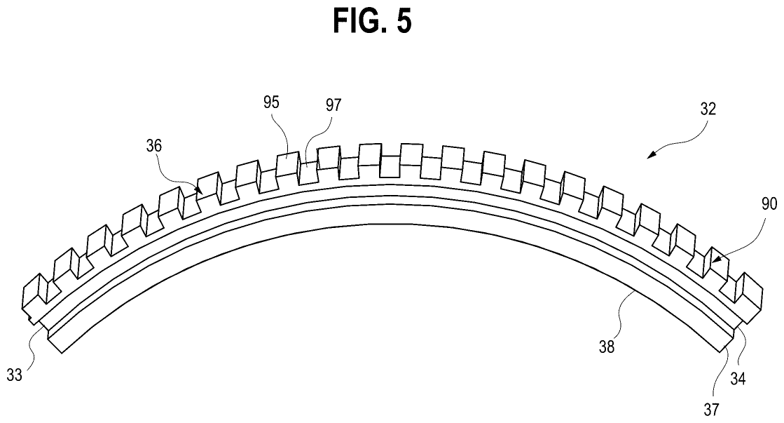

The stabilization system 1 includes a supporting rail or frame, portions of which are illustrated at 30, which may be secured to a patient's face by a suitable attachment apparatus, for example an elastomeric strap, hook and loop fasteners (Velcro©), and the like (not shown). As shown in FIG. 5, the rail has a generally symmetrical curved configuration or body portion contoured to permit it to conform to a patient's face when it is secured in position. It may be formed of plastic, rubber, nonferromagnetic metal, composite material, or other suitable materials having the desired physical properties for the application. The rail includes a curved body 32 which by way of example in the embodiment shown is in the shape of an I-beam, having first and second ends 33, 34, an outer or first rail member 35 having an upper or outer surface 36 facing away from the patient and an inner or second rail member 37 including a face or surface 38 which has a layer of stationary cushioning foam 40 facing toward the patient affixed thereto. An exemplary layer of cushioning foam is illustrated in FIG. 4 and, in the embodiment shown, includes an elongate rectangular body 41 configured to overlay surface 38 of the second rail member, the elongate rectangular body further having a face or surface 42 adapted to be affixed to surface 38 by suitable fastening systems such as adhesives, double sided tape, and the like.

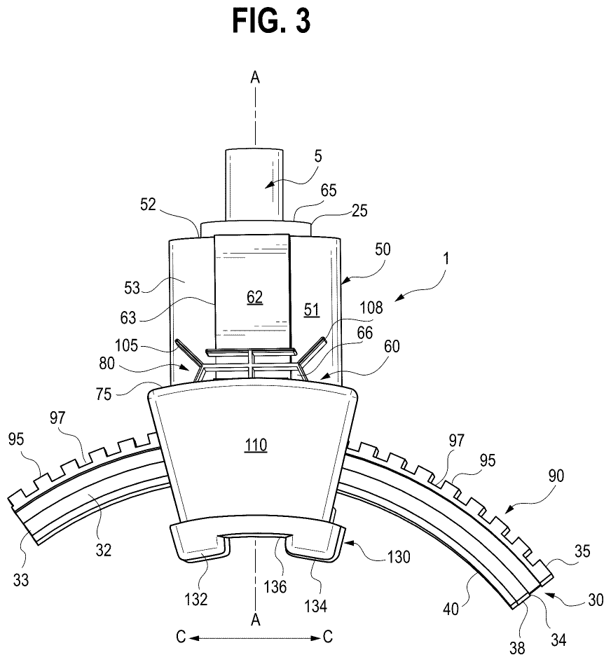

Referring again to FIG. 1 and also to FIG. 3, the stabilization system 1 includes the generally cylindrically-shaped tower structure or clamshell-type clamping member 50 adjustably and operatively connected to the rail 30 is illustrated in greater detail. The tower structure is configured to interact in clamping engagement with the continuous sidewall 14 of the airway device via the interlock collar 25 which is removably and adjustably positioned in and adjustably connected to the stabilization system via the tower structure and cooperates therewith to prevent clinically significant movement of the proximal end of the airway device with respect to the vocal cords of the patient. The tower structure/clamping member 50 extends in a substantially perpendicular direction from the outer surface 35 of the rail 30 coaxially along axis A-A in a direction away from the patient's face. The tower structure includes a cylindrical body portion 51 having inner and outer surfaces 52 and 53 respectively extending circumferentially about and coaxially along axis A-A.

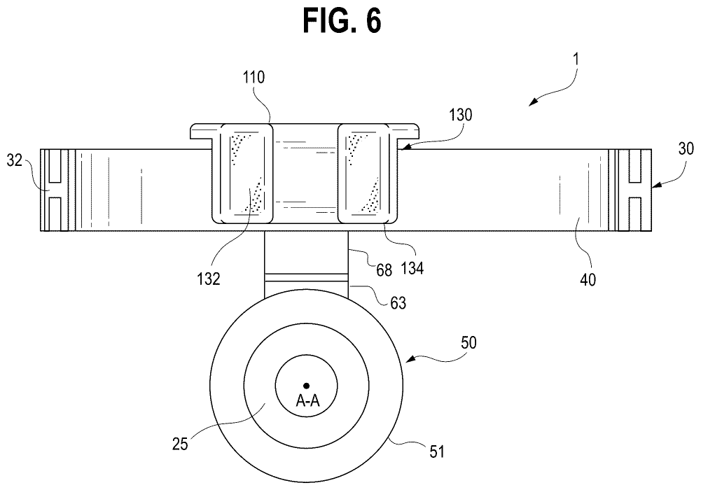

Referring to FIGS. 1, 3, 5, 6, and 7, the lateral position adjustment mechanism 60 of the present invention are specifically described. A bracket, clip, or mounting arm 61, is shown. In the instant embodiment, the bracket is L-shaped; however, it is to be understood that other configurations, for example, U-shaped, may also be used without departing from the scope of the present invention. The L-shaped bracket includes a first leg member 62 having a body 63 and first and second ends 65, 66 the body being operatively connected to the outer surface 53 of the tower structure 50 and extending in a direction parallel to the axis A-A and, at the second end 66 thereof, to a first end 67 of a rectangularly shaped body portion 68 of a second leg member 70 extending perpendicularly therefrom in a laterally outwardly direction. The body portion of the second leg member 70 includes a flat surface 75 lying in a plane that is perpendicular to axis A-A. A release attachment mechanism 80 (FIGS. 3, 7 and 8) is secured to the flat upper surface 75. A curved track 90 is operatively connected thereto or formed integrally on the upper surface 35 of the rail 30 and includes a plurality of spaced apart teeth 95 extending intermediate the first and second ends 33, 34 of the rail body 32 in a direction generally perpendicular to the axis A-A. Each of the plurality of teeth is separated from an adjacent one of the plurality of teeth by a space or recess 97, the plurality of teeth and recesses being adapted to releasably engage one or more downwardly extending mating teeth 99 operatively pivotally connected to release attachment mechanism 80. The release attachment mechanism is structured and arranged to moveably fit over the curved track 90 and be releasably secured thereto by way of example and not of limitation outwardly biased ears, wings or levers 105, 108 pivotally connected to the release attachment mechanism. The wings or levers may be manipulated by squeezing them together manually, each of which flexes apart a lower portion of a respective one of the plurality of teeth, thus releasing them from recesses 97. Squeezing the ears or levers and releasing the teeth allows adjustment of the lateral position of the securing apparatus, and therefore the tower, the airway device secured thereby, and a pillow or cushioning device, as will be described in greater detail below, in the direction of the arrow C-C on a patient.

A channel member 110 having a first leg 112 is operatively connected to edge 115 of the rectangular shaped body 68 and extends inwardly therefrom toward the patient. In the embodiment shown, the channel member is L-shaped; however, it is to be understood that other configurations, for example, U-shaped, may also be used without departing from the scope of the present invention.

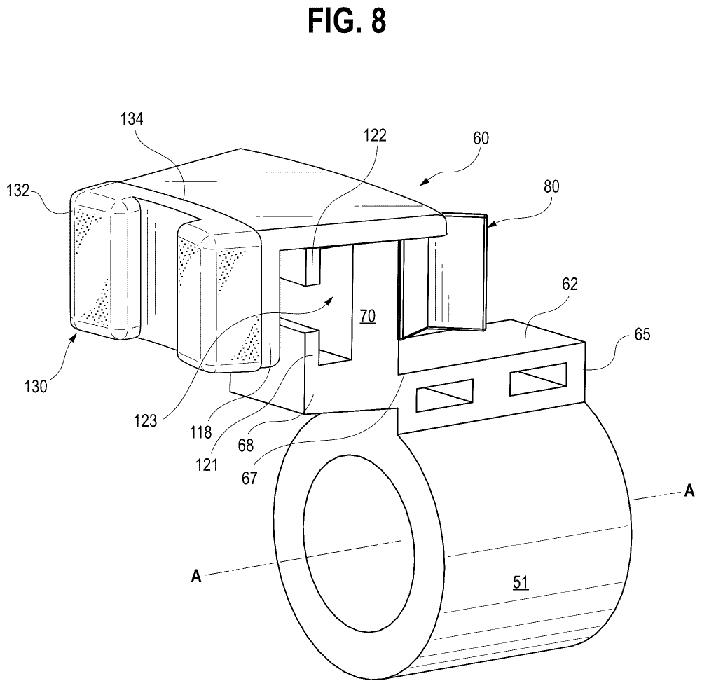

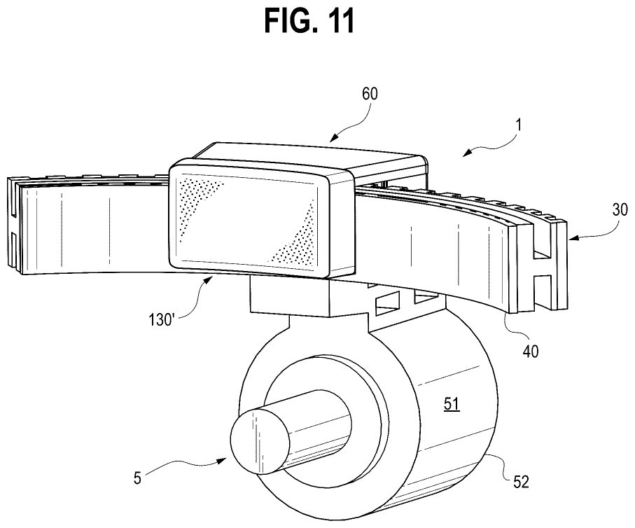

As best shown in FIGS. 1 and 8, the L-shaped channel member further includes a second leg 118 operatively connected to or formed integrally with and perpendicular to the first leg 112. The second leg extends in a downward direction perpendicular to axis A-A adjacent the lower or bottom supporting portion 38 of the rail body 32 and cushioning layer 40. The second leg further includes an outer surface 120 which faces toward the patient and to which a pillow or cushioning member 130 is affixed. The cushioning member is adapted to be selectively positioned in contacting engagement with a patient's upper lip and is adapted to minimize the magnitude of and the time during which the airway device and securement apparatus exert pressure on any specific region of the patient's upper lip. In the embodiments of FIGS. 1-9, the cushioning member 130 is in the form of a split cushion or pillow having a pair of spaced-apart raised portions 132, 134 for positioning in contacting engagement with a patient's upper lip, the raised portions being separated by a recessed portion 136. The split cushion or pillow configuration may find application in situations where a patient has an upper lip injury or area of irritation that would be aggravated by the use of a solid, rectangularly shaped pillow, such as the pillow 130′ of FIG. 11. In the latter embodiment, the pillow or cushion does not include the recessed portion 136 of FIGS. 1-9. However, it is to be understood that the pillow can be any of a number of shapes and sizes.

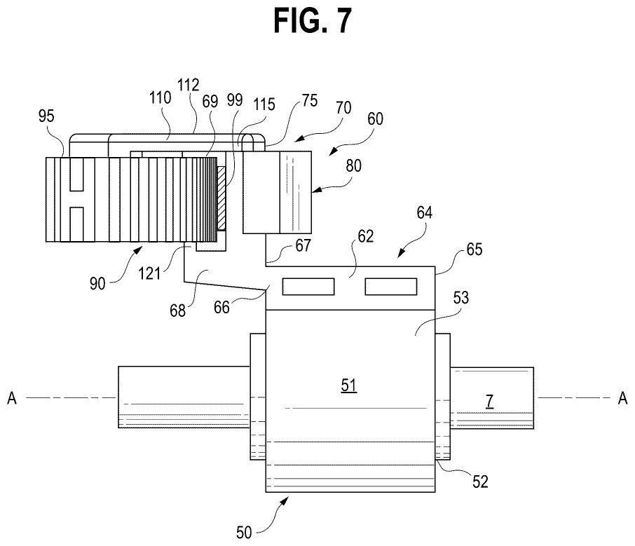

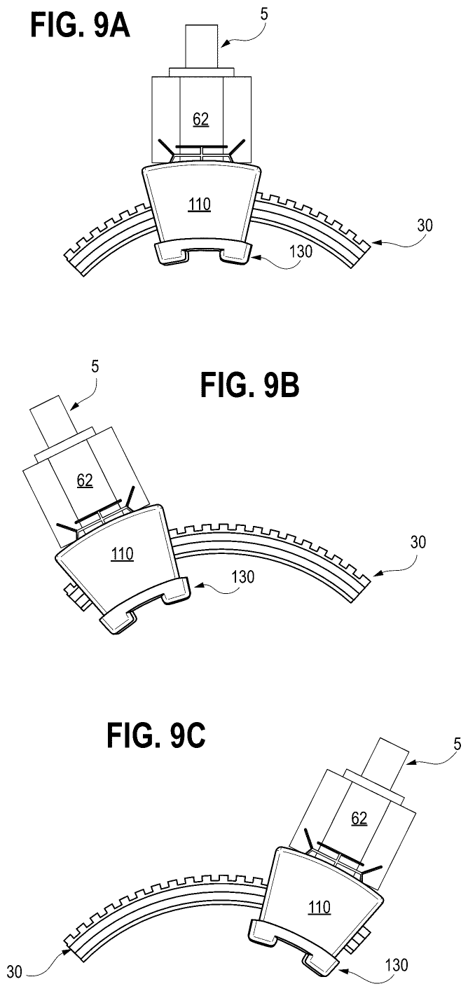

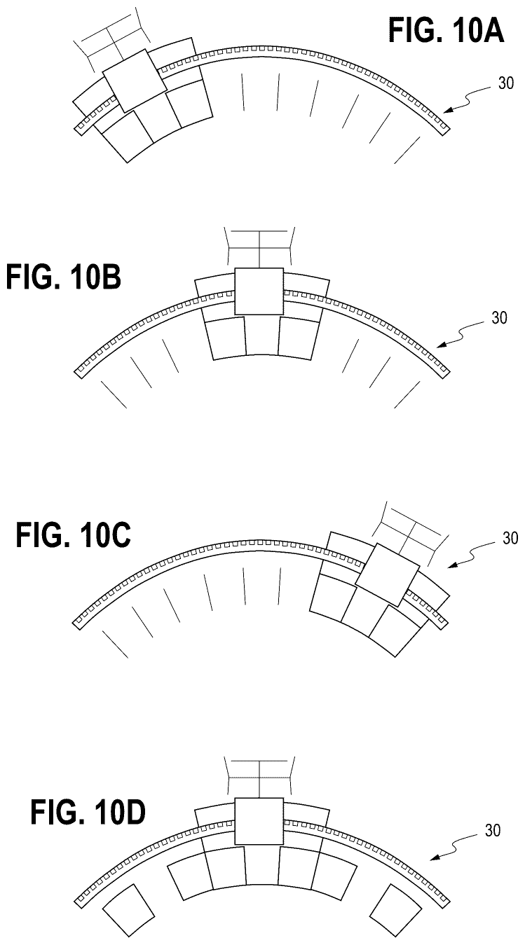

Referring now to FIGS. 7 and 8, the features of the rectangularly shaped body portion 68 of the second leg member 70 are shown in greater detail. Body portion 68 includes a vertical member 121 extending upwardly from the rectangularly shaped body portion 68 of the second leg member 70 in a direction which is perpendicular to the axis A-A. The second leg member 70 further includes a second rectangularly shaped body portion 69, which is not visible in the perspective views discussed above. Body portion 69 is located in a spaced apart, juxtaposed position relative to body portion 68 and parallel thereto. It extends outwardly from member 70 in a direction toward a patient's face and includes a downwardly extending member 122 which cooperates with upwardly extending member 121 to form a cavity 123 having an access aperture 124 adapted to slidably receive track 30 therein. As the stabilization system securing an airway device is moved to different positions on a patient's upper lip as shown in FIGS. 9a-9c, the L-shaped bracket or mounting arm 60 slides easily along the rail 30 and track 35 located in cavity 123 until the tabs 105 and 108 are released, thus locking the system in place in a different selected location relative to the patient's upper lip. FIGS. 10a-10d illustrate the split cushion's footprints at left, center and right locations on a patient's upper lip, which show the relief provided to any one location after the system is repositioned in a timely manner to avoid irritation and/or injury to a patient's upper lip during the ventilation process.

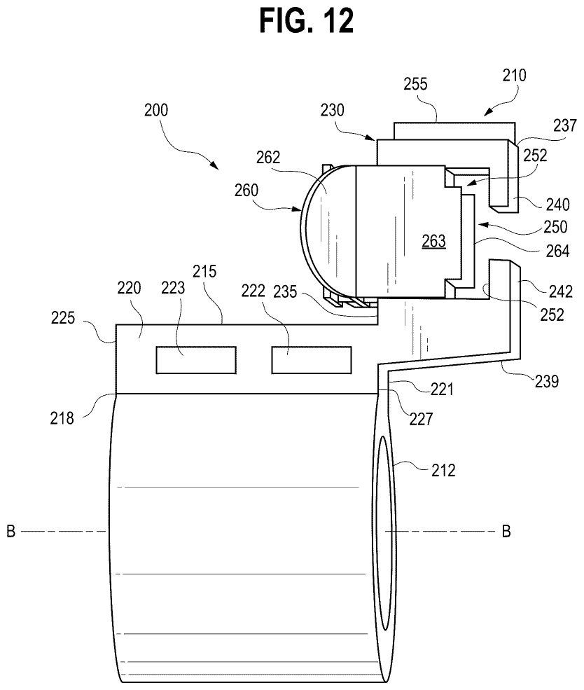

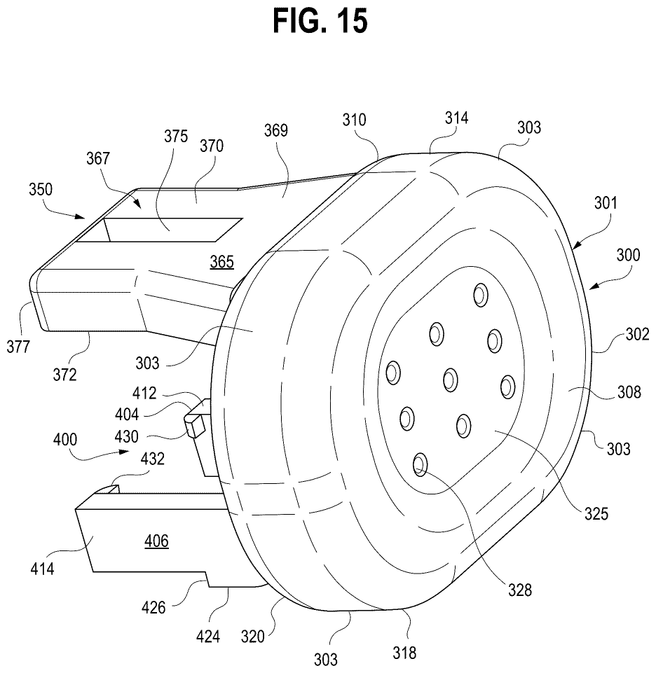

Referring now to FIGS. 12-14, portions of an airway device securement and stabilization system, again referred to herein for purposes of brevity as “the stabilization system”, are depicted generally at 200 in accordance with another embodiment of the present invention. The system includes a securing mechanism 210 operatively connected to a generally cylindrically-shaped tower structure or clamshell-type clamping member 212. As described above with respect to the embodiment of FIG. 1, the tower structure is configured to interact in clamping engagement with a continuous sidewall 213 of airway device 216 via an interlock collar 217 which is removably and adjustably positioned in and adjustably connected to the stabilization system via the tower structure and cooperates therewith to prevent clinically significant movement of a proximal end of the airway device with respect to the vocal cords of the patient. In the same manner as shown in FIG. 1, the securing mechanism 210 is adapted to operatively connect the tower structure, an airway device secured therein and a pillow or cushioning member such as cushioning member 300 illustrated in FIG. 15 et. seq., to a curved track 90 which is formed in the upper surface 35 of the rail 30 and includes a plurality of spaced apart teeth 95 extending intermediate the first and second ends 33, 34 of the rail body 32 in a direction generally perpendicular to the axis B-B.

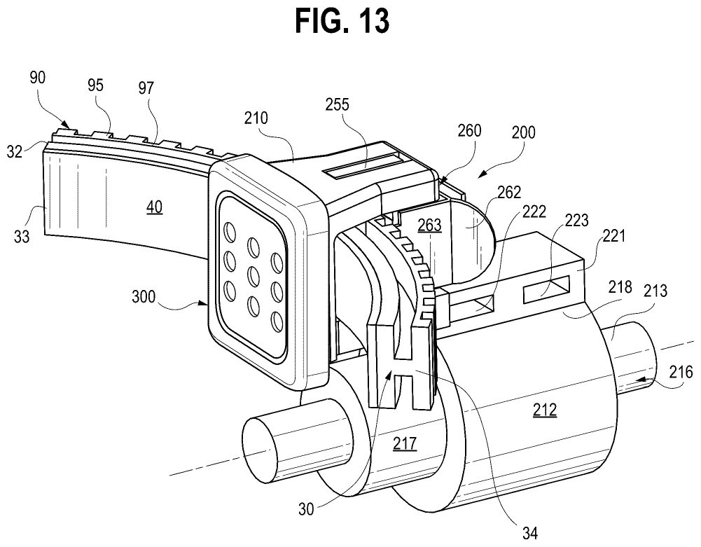

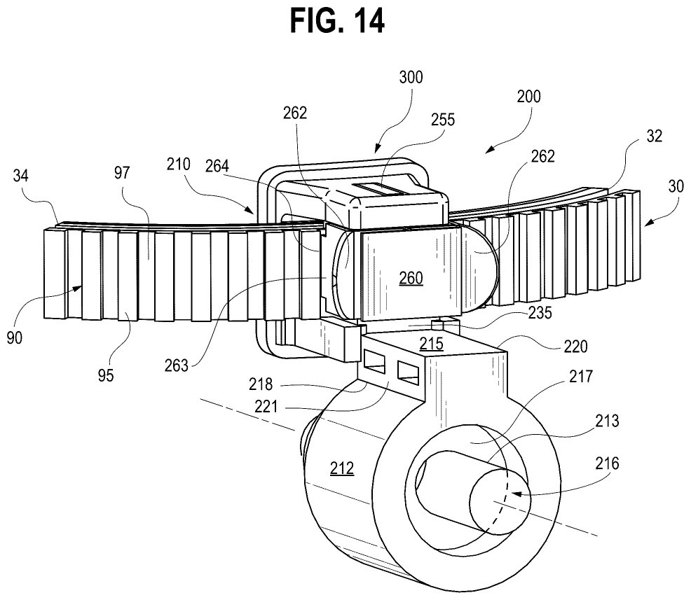

In the embodiment of FIG. 12, the securing mechanism 210 includes a rectangularly shaped beam member 215 connected at a lower or bottom surface 218 to the cylindrically-shaped tower structure 212, an upper or top surface 215, a pair of spaced apart parallel sides 220, 221 extending intermediate the upper and lower surfaces in a direction perpendicular thereto, a pair of apertures 222, 223 extending intermediate the spaced apart parallel sides 220, 221 in a direction perpendicular thereto, and first and second parallel ends 225, 227 which extend intermediate and perpendicular to the upper and lower surfaces and the a pair of spaced apart parallel sides. The securing mechanism further includes a c-shaped channel member 230 having a vertically extending first leg 235 which is operatively connected to the second end 227 of the rectangular shaped beam member 215. A pair of oppositely disposed L-shaped members 237, 239 extend inwardly therefrom toward the patient in a direction parallel to the axis B-B, each terminating in an end portion 240, 242 which extend perpendicularly toward one another but terminating short of actually touching, thereby forming a gap or access slot 250 extending into an open space or aperture 252 therebetween. The access slot and aperture are adapted to releasably receive a curved track such as track 90 shown in FIGS. 13 and 14 on which the securing mechanism is selectively positioned. Tab 255 operatively connected to L-shaped member 237 is adapted to releasably receive a portion of an attachment mechanism in accordance with another embodiment of the invention as will be described below with respect to FIGS. 14-16.

A release attachment mechanism 260 is secured to the vertically extending first leg 235 of c-shaped channel member 230 and is structured and arranged to moveably fit over the linear track 90 (FIG. 1) and be releasably secured thereto by way of example and not of limitation outwardly biased ears, wings or levers 262. Each wing includes a body portion 263 that terminates in an elongated flat tooth-like projection 264 adapted to releasably engage one of the plurality of recesses 97 formed intermediate teeth 95 of the track or rail 90. The wings or levers may be manipulated by squeezing them together manually, each of which flexes apart the respective elongated flat tooth-like projection thereof, thus releasing them from recesses 97. Squeezing the wings or levers and releasing the teeth allows adjustment of the lateral position of the securing apparatus, and therefore the tower, the airway device secured thereby, and a pillow or cushioning device, along the track as described above.

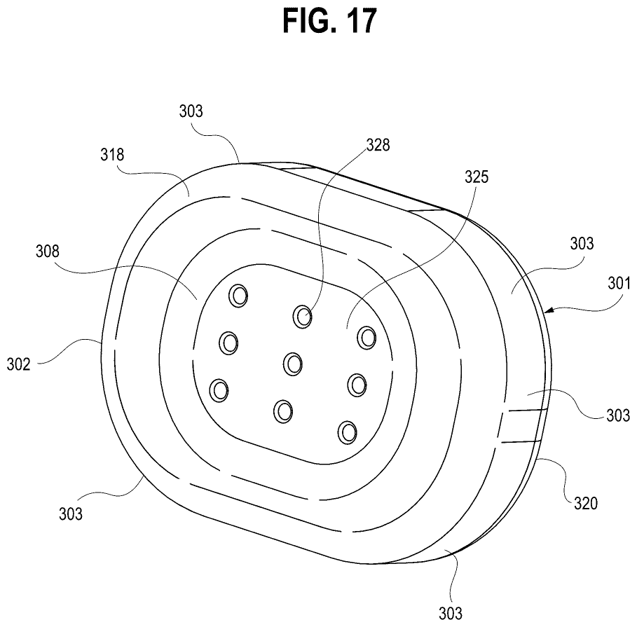

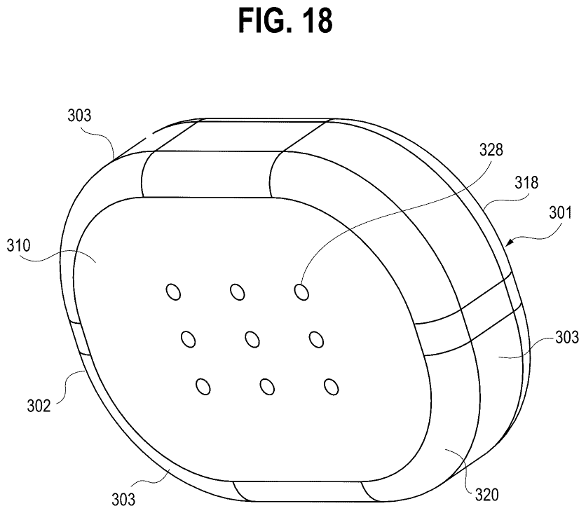

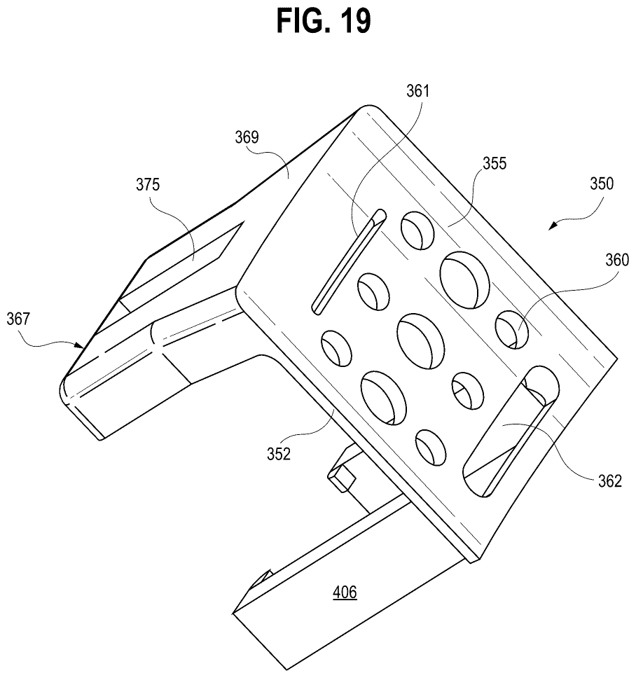

FIGS. 15-20 illustrate the details of a pillow or cushion assembly 300 operatively connected to an attachment mechanism or shuttle 350 in accordance with another embodiment of the present invention. By way of illustration and not of limitation, cushion 300 includes a pillow 301 having a rectangular body 302 with rounded corners 303, a top surface or face 308 and a bottom or back surface 310 respectively separated or spaced apart by a side surface 314 which extends circumferentially around the body intermediate the top and bottom surfaces and is separated therefrom by upper and lower rounded edges 318, 320 respectively. As best shown in FIG. 17, the top face 308 is slightly recessed or dish-shaped, transitioning gradually from the rounded edge 318 to a saucer-like rectangular recessed surface area 325 having a plurality of perforations 328 formed therein which act as air release valves and also act to allow some air to circulate onto the patient's skin. In the embodiment depicted in FIG. 18, the bottom surface or face 310 is flat or straight; however, as will be discussed in greater detail below, it is to be understood that the back surface of the pillow may be curved to fit a shuttle which has a curved top surface.

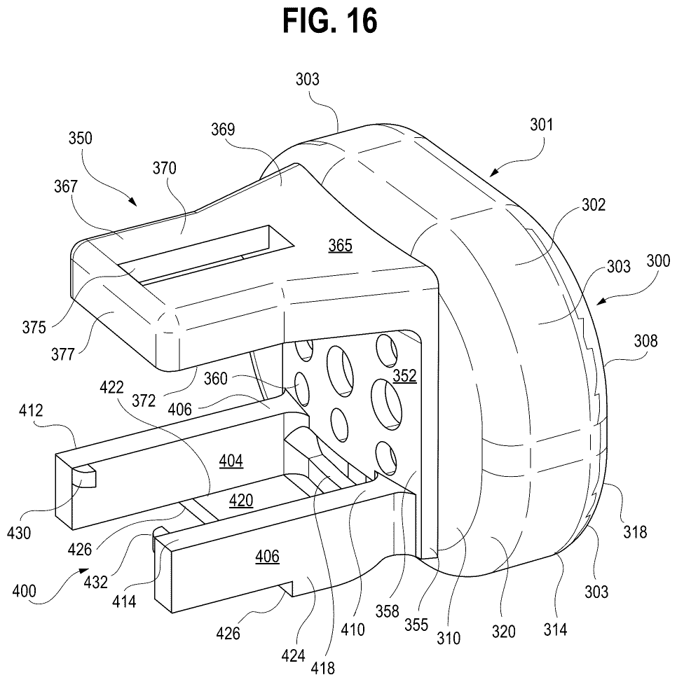

Referring to FIGS. 16, 19, and 20, the attachment mechanism or shuttle 350 has a configuration similar to that of a pedestal, table, or bench laid on its side. It includes a top table-like member 352 having top and bottom surfaces 355, 358, the top table-like member further including a plurality of spaced apart apertures 360 extending intermediate the top and bottom surfaces which reduce the weight of the pedestal and allow air to be circulated, thereby cooperating with plurality of perforations 328 formed the body 302 pillow 301 in minimizing moisture accumulation under the cushion, decreasing the risk of maceration of the skin, and enhancing the patient's comfort while the apparatus is position. In the embodiment shown, the apertures 360 are uniformly distributed in a matrix-type arrangement; however, it is to be understood that a random distribution pattern may also be employed without departing from the scope of the present invention. The top table-like member 352 further includes a pair of oppositely disposed, transversely extending slots 361, 362 formed therein and extending intermediate the top and bottom surfaces 355, 358, the slots being adapted to releasably secure various embodiments of a cushion or pillow in response to the needs of a particular patient or application of the airway device securement and stabilization system. The top surface 355 is secured to the bottom surface 310 of the cushion by suitable attachment means such as medical adhesive, double-sided tape, hook and loop fasteners or other suitable securing methods.

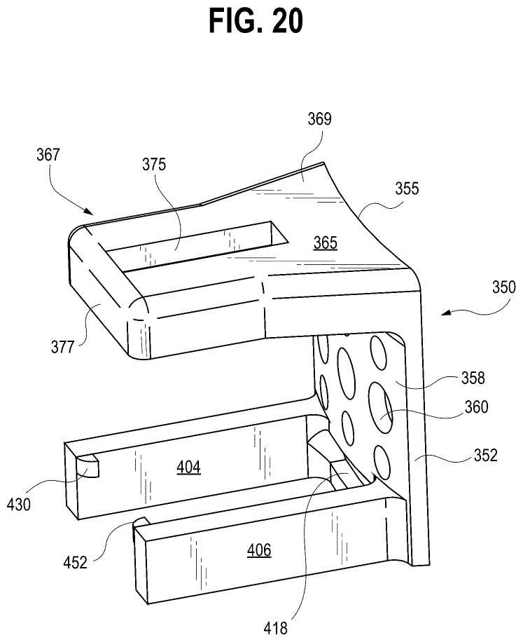

As best shown in FIGS. 16 and 20, the attachment mechanism includes an upper leg 365 secured to or formed integrally with the top table-like member 352 and extending perpendicularly therefrom in a distal direction, that is, in a direction away from the patient's face. The upper leg is generally rectangularly shaped having a flat body 367 with a slightly flared upper end 369, a top surface 370, a bottom surface 372, and a rectangular slot 375 extending intermediate the top and bottom surfaces terminating in and end cap 377. The slot is structured and arranged to be releasably received over the tab 255 of the securing mechanism 210.

The attachment mechanism 350 further includes a lower attachment frame assembly 400 comprising a pair of spaced apart legs 404, 406, each leg having a first end 408, 410 and a second end 412, 414 respectively, the first ends being interconnected by a spacer or support member 418 extending therebetween and secured thereto. As illustrated in FIG. 16, plank-shaped reinforcing member 420 having first and second ends 422, 424 operatively connected to legs 404 and 406 may optionally be included as part of the structure of the frame assembly to further strengthen the frame assembly. The reinforcing member has a bottom edge 426 which aids in the positioning of the pillow or cushion 300 and attachment mechanism 350 in the access slot 250 extending into the open space or aperture 252 of the securing mechanism 210 shown in FIG. 12. A pair of tabs 430, 432 secured to the respective second ends 412, 412 of the spaced apart legs 404, 406 interact with the aperture 252 to releasably locate and secure the attachment mechanism therein.

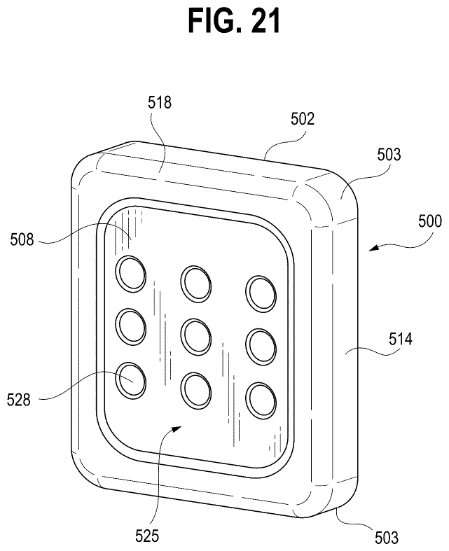

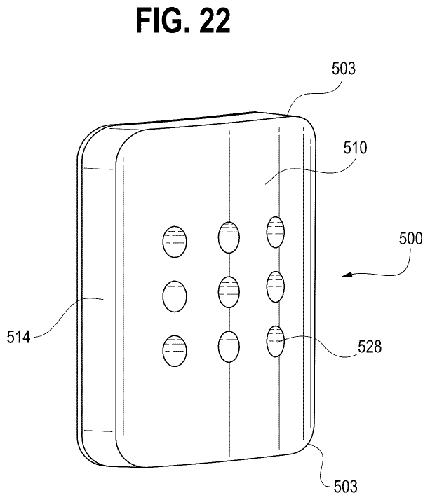

FIGS. 21 and 22 illustrate a pillow or cushion 500 adapted to be operatively connected to an attachment mechanism or shuttle 350 as depicted in FIG. 16 in accordance with another embodiment of the present invention. Cushion 500 includes a square body 502 with rounded corners 503, a top surface or face 508 and a bottom or back surface 510 separated or spaced apart by a side surface 514 which extends circumferentially around the body intermediate the top and bottom surfaces. A rounded edge 518 disposed intermediate the top and side surface separates the two surfaces. The top face 508 is slightly recessed or dish-shaped, transitioning gradually from the rounded edge 518 to a saucer-like square recessed surface area 525 having a plurality of perforations 528 formed therein which act as air release valves and also act to allow some air to circulate onto the patient's skin. As discussed with respect to the embodiment depicted in FIGS. 17 and 18, the bottom surface or face 510 may be either flat or straight; or it may be curved to fit a shuttle which has a curved top surface.

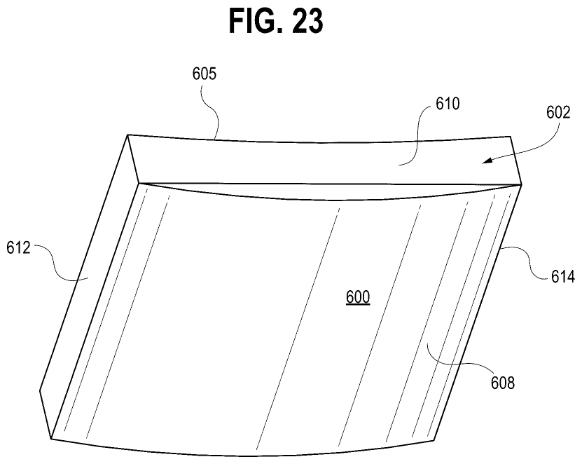

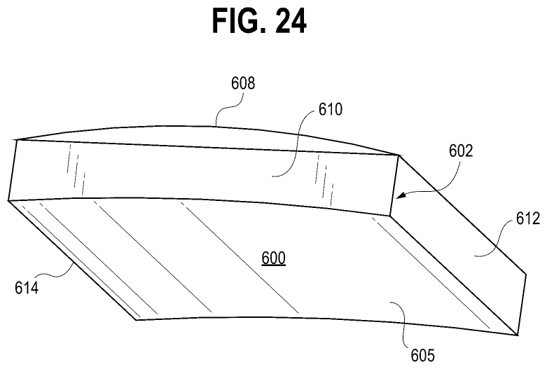

FIGS. 23 and 24 illustrate yet another embodiment of a pillow or cushion 600 which is formed of a single piece of foam, silicone, or other resilient materials suitable for the application or with an internal cavity or chamber filled with air or liquid to allow for pressure to be redistributed on a patient's upper lip in response to movement of an airway device and a securement and stabilization system to which it is attached to different positions on the patient. Cushion 600 includes a square body 602, a top surface or face 605, and a bottom or back surface 608 separated or spaced apart by a side surface 610 which extends circumferentially around the body intermediate the top and bottom surfaces. The top face 605 is laterally inwardly recessed intermediate first and second vertically extending edges 612 and 614 to permit it to conform to a patient's face when it is secured in position thereon.

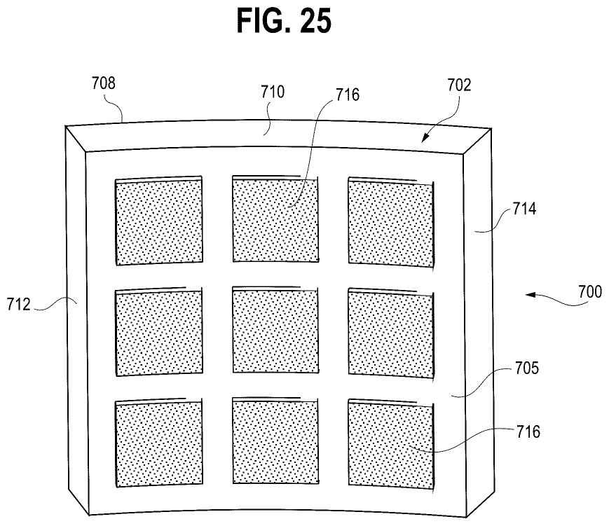

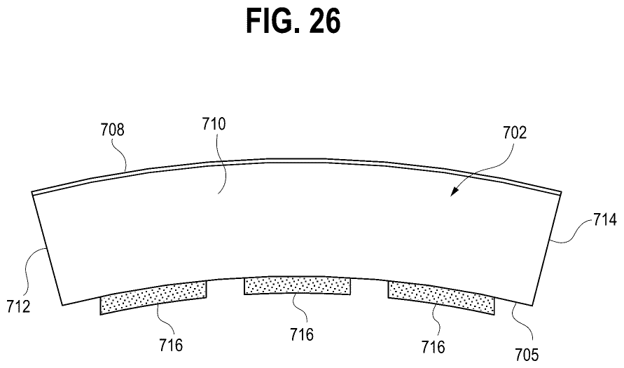

Still another embodiment of a pillow or cushion of the present invention is depicted at 700 in FIGS. 25 and 26. Similar in construction and configuration to the a pillow or cushion 500 shown in FIGS. 23 and 24, pillow 700 is formed of a single piece of foam, silicone, or other resilient materials suitable for the application or with an internal cavity or chamber filled with air or liquid to allow for pressure to be redistributed on a patient's upper lip in response to movement of an airway device and a securement and stabilization system to which it is attached to different positions on the patient. When viewed from the front as in FIG. 25, cushion 700 presents as a truncated pyramid-shaped body 702 having a top or front surface or face 705, and a bottom or back surface 708 separated or spaced apart by a side surface 710 which extends circumferentially around the body intermediate the top and bottom surfaces. Both the top and bottom surfaces 705 and 708 are laterally inwardly recessed intermediate first and second vertically extending edges 712 and 714 to permit the pillow to conform to a patient's face when it is secured in position thereon, the back surface 708 being slightly larger in size than the front face 705, thus resulting in the truncated configuration of the body 702. The front face 705 further includes a plurality of uniformly distributed protuberances 716 extending outwardly a predetermined distance from the front face, each of which, in the embodiment shown, are in the form of a square. However, the protuberances may take the form of a semi-circle, a semi egg-shaped body and the like without departing from the scope of this invention, the primary function thereof being to permit air circulation intermediate the pillow and the patient's face.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. An adjustable airway device securement and stabilization system for stabilizing and securing an airway device to maintain an airway to a patient's lungs, the patient having a face, a mouth, an upper and a lower lip, a nose, an oral cavity, a throat, vocal cords or larynx, a trachea having a length and forming an air passageway or airway in the patient, and a carina defining a point at which the trachea bifurcates into a left and a right bronchial tube, the airway stabilization system comprising:
an airway device having a flexible elongate body extending along a longitudinal axis and having a length, the flexible body including a continuous sidewall having an internal surface and an external surface extending between a proximal (patient-end) and a distal (machine-end) portion thereof, thereby forming a hollow conduit or airway;
a supporting rail or frame adapted to be secured to the patient;
an attachment apparatus operatively connected to the supporting rail or frame and adapted to releasably secure the supporting rail or frame to the patient;
an adjustable interlock collar selectively positioned on and in operative securing engagement with the airway device;
a securing apparatus or stabilizer adjustably secured to the supporting rail or frame and extending outwardly therefrom along a longitudinal axis which extends coaxially with the longitudinal axis of the airway device in a direction away from a patient's face, the securing apparatus or stabilizer being adapted to interact in clamping engagement with the adjustable interlock collar;
a cushioning member attachment mechanism operatively connected to the securing apparatus or stabilizer and to the supporting rail or frame allowing movement of the cushioning member attachment mechanism along the supporting rail; and
a cushioning member adapted to be selectively positioned in contacting engagement with a patient's upper lip whereby the magnitude of and the time during which the airway device and securement apparatus exert pressure on any specific region of the patient's upper lip is minimized.

2. The adjustable airway device securement and stabilization system of claim 1 wherein the supporting rail or frame includes a curved body portion adapted to conform to a patient's face, the body portion having first and second ends, an outer or first rail member having an upper or outer surface facing away from the patient and an inner or second rail member having a face or surface facing toward the patient, and a layer of cushioning foam operatively connected to the face or surface of the second rail member.

3. The adjustable airway device securement and stabilization system of claim 2 wherein the upper or outer surface of the supporting rail or frame includes a curved track operatively connected thereto or integrally formed therewith, the curved track including a plurality of spaced apart teeth extending intermediate the first and second ends of the rail body in a direction perpendicular to the longitudinal axis of the airway device.

4. The adjustable airway device securement and stabilization system of claim 3 wherein each of the plurality of teeth is separated from an adjacent one of the plurality of teeth by a space or recess.

5. The adjustable airway device securement and stabilization system of claim 4 further including a lateral positioning adjustment mechanism adapted to selectively position the cushioning member laterally to different positions on the patient's upper lip.

6. The adjustable airway device securement and stabilization system of claim 5 wherein the lateral positioning adjustment mechanism includes a bracket having a first leg member, the leg member having a body, the body including first and second ends, the body being operatively connected to an outer surface of the securing apparatus or stabilizer and extending in a direction parallel to the stabilizer's longitudinal axis, the second end of the body being operatively connected to a first end of a rectangularly shaped body portion of a second leg member extending perpendicularly therefrom in a laterally outwardly direction.

7. The adjustable airway device securement and stabilization system of claim 6 further including a release attachment mechanism, the release attachment mechanism being adapted to be movably positioned over the curved track and releasably connected thereto by one or more downwardly extending mating teeth operatively pivotally connected to the release attachment mechanism.

8. The adjustable airway device securement and stabilization system of claim 7 wherein each of the one or more downwardly extending mating teeth is operatively connected to an outwardly biased lever, each outwardly biased lever being pivotally connected to the release mechanism.

9. The adjustable airway device securement and stabilization system of claim 8 wherein the lateral positioning adjustment mechanism further includes a vertical member extending upwardly from the rectangularly shaped body portion of the second leg member in a direction perpendicular to the stabilizer's longitudinal axis, the second leg member further including a second rectangularly shaped body portion located in a spaced apart, juxtaposed position relative to the rectangularly shaped body portion of the second leg member and parallel thereto and extending outwardly from the vertical member in a direction toward a patient's face, the second rectangularly shaped body portion including a downwardly extending member adapted to cooperate with the vertically extending member to form a cavity having an access aperture adapted to slidably receive the curved track therein.

10. The adjustable airway device securement and stabilization system of claim 1 wherein the cushioning member is a split cushion or pillow having a pair of spaced-apart raised portions adapted to be positioned in contacting engagement with a patient's upper lip, the raised portions being separated by a recessed portion.

11. The adjustable airway device securement and stabilization system of claim 1 wherein the cushioning member is a solid, rectangularly shaped cushion or pillow.

12. The adjustable airway device securement and stabilization system of claim 1 wherein the cushioning member comprises a cushion assembly, the cushion assembly including a cushion or pillow operatively connected to an attachment mechanism or shuttle, the shuttle having a top table-like member including top and bottom surfaces, the top table-like member further including a plurality of spaced apart apertures extending intermediate the top and bottom surfaces.

13. The adjustable airway device securement and stabilization system of claim 12 wherein the attachment mechanism or shuttle includes an upper leg operatively connected to or formed integrally with the top table-like member and extending perpendicularly therefrom in a distal direction away from a patient's face, the upper leg having rectangularly shaped flat body including a flared upper end, a top surface, a bottom surface, and a rectangular slot extending intermediate the top and bottom surfaces and terminating in and end cap.

14. The adjustable airway device securement and stabilization system of claim 13 wherein the rectangular slot is adapted to be releasably received over a tab portion of the securing mechanism.

15. The adjustable airway device securement and stabilization system of claim 14 wherein the top table-like member has a curved upper surface.

16. The adjustable airway device securement and stabilization system of claim 15 wherein the cushion or pillow includes a rectangular body having rounded corners, a top surface or face, and a bottom or back surface, the top surface and the bottom surface being separated or spaced apart by a side surface extending circumferentially around the body intermediate the top and bottom surfaces and separated therefrom by upper and lower rounded edges.

17. The adjustable airway device securement and stabilization system of claim 16 wherein the top face is recessed or dish-shaped, transitioning gradually from the upper rounded edge to a saucer-like rectangular recessed surface area having a plurality of perforations formed therein, the plurality of perforations being adapted to allow air to circulate onto a patient's skin.

18. The adjustable airway device securement and stabilization system of claim 17 wherein the attachment mechanism or shuttle includes a lower attachment frame assembly comprising a pair of spaced apart legs, each leg having a first end and a second end respectively, the first ends being interconnected by a spacer or support member extending therebetween and secured thereto.

19. The adjustable airway device securement and stabilization system of claim 18 wherein the cushion or pillow includes a square body.

20. The adjustable airway device securement and stabilization system of claim 19 wherein the cushion or pillow includes a truncated pyramid-shaped body having a top or front surface or face, and a bottom or back surface separated or spaced apart by a side surface which extending circumferentially around the body intermediate the top and bottom surfaces, the top and bottom surfaces each being laterally inwardly recessed intermediate first and second vertically extending edges, the top surface further including a plurality of uniformly distributed protuberances extending outwardly a predetermined distance from the front face.
